(19) United States
(12) Patent Application Publication
(10) Pub. No.: US 2012/0133363 A1
(43) Pub. Date: May 31, 2012

(54) MAGNETIC RESONANCE COMPATIBLE AND SUSCEPTIBILITY-MATCHED APPARATUS AND METHOD FOR MR IMAGING & SPECTROSCOPY

(75) Inventors: Thomas Harold Mareci, Gainesville, FL (US); Paul Richard Carney, Gainesville, FL (US); Garrett William Astary, Gainesville, FL (US); Hector Sepulveda, Las Vegas, NV (US); Michele Viola Manuel, Gainesville, FL (US); Charles Robert Fisher, Gainesvile, FL (US)

(73) Assignee: UNIVERSITY OF FLORIDA RESEARCH FOUNDATION, INC., Gainesville, FL (US)

(21) Appl. No.: 13/389,289

(22) PCT Filed: Aug. 9, 2010
(86) PCT No.: PCT/US10/44905

§ 371 Date: Feb. 7, 2012

Related U.S. Application Data
(60) Provisional application No. 61/232,288, filed on Aug. 7, 2009.

(51) Int. Cl. G01R 33/48; A61M 25/00; A61B 5/04

(52) U.S. Cl. 324/309; 600/373; 604/264

(57) ABSTRACT

Electrodes, infusion cannula, and interventional MRI instrumentation, are constructed with multiple layers or mixtures of metals or alloys that allow the diamagnetic behavior of some metals to combine with the paramagnetic behavior of others, wherein the devices have a magnetic susceptibility which is close to that of the material, for example body tissue, being im The material may thereby be imaged using MRI with resultant images having greatly reduced distortion. Optimal metal composites are determined through mathematical modeling and measurements. In particular, MR compatible susceptibility-matched electrodes may be used for stimulation and for acquiring electroencephalography (EEG) data before, during and after MR image and spectroscopy measurements, with a significant reduction in distortion of the resulting images and spectra. In accordance with the invention, these electrodes may further be incorporated into micro-electrode arrays. In addition, MR compatible susceptibility-matched cannula can implanted before, during and after MR image and spectroscopy measurements, with a significant reduction in distortion of the resulting images and spectra.

### CROSS REFERENCE TO RELATED APPLICATIONS

This application claims the benefit of priority to U.S. Provisional Application Ser. No. 61/232,288, filed Aug. 7, 2009, the contents of which is hereby incorporated by reference in its entirety.

### FIELD OF THE INVENTION

The invention relates to minimizing magnetic resonance image and spectral distortions introduced near the interface between a device and the surrounding material.

### BACKGROUND OF THE INVENTION

Known stimulating/recording electrodes typically consist of stainless steel or tungsten wires, with an insulating layer of polyimide or Teflon (a registered trademark of I.E. Dupont De Nemours and Co., Wilmington, Del.). These electrodes have magnetic susceptibility properties that are significantly different than biological tissue, and this leads to fluctuations in the local magnetic field at the tissue-electrode interface. Local magnetic field fluctuations can result in signal loss and affect the spatial encoding of the MR image, resulting in image distortions. Alternative materials have been proposed [2][3]; however, a need remained for improvements in image quality and accuracy, with a reduction of distortions, which include perturbations, artifacts, and an increase in the apparent dimensions of an introduced device.

Fused-silica cannulas are additionally used during MRI, predominately when direct tissue infusions are observed in real-time. Fused-silica has a magnetic susceptibility that is similar to surrounding tissue, and thus cannula constructed out of fused-silica do not cause substantial image distortions. Unfortunately, fused-silica exhibits poor conductivity, which limits its use in a recording electrode. Moreover, fused-silica is very flexible, and can therefore be difficult to control during insertion.

### SUMMARY OF THE INVENTION

The invention provides non-magnetic, susceptibility-matched metal devices for magnetic resonance imaging (MRI) and MR spectroscopy, that have magnetic susceptibility properties matched to materials in which the device is used (e.g. biological tissues). Susceptibility-matching the metal devices to the surrounding material reduces perturbations in the local magnetic field, thus minimizing MR image and spectral distortions introduced near the interface between device and the surrounding material.

It should be understood that non-magnetic and magnetic susceptibility are different terms. As stated in Schenck [1], “Materials with the first kind of magnetic field compatibility are such that magnetic forces and torques do not interfere significantly when the materials are used within the magnetic field of the scanner; materials with the second kind of magnetic field compatibility meet the more demanding requirement that they produce only negligible artifacts within the MR image and their effect on the positional accuracy of features within the image is negligible or can readily be corrected”. This invention is primarily directed to problems relating to the “second kind” of magnetic field compatibility.

Magnetic susceptibility (χ) describes how the magnetization in a material is proportional to an applied magnetic field. As the applied magnetic field permeates through a substance with spatially varying susceptibility, local variations in the magnetic field will occur at the interface between regions with different susceptibilities. Surgical clips, brain sinuses, blood vessels and in-dwelling cannula are a few examples of objects in the body that can cause variations in the local magnetic field.

In terms of susceptibility, materials can be classified into four categories:

(1) diamagnetic (χ<0)

(2) non-magnetic (χ=0)

(3) paramagnetic (χ>0)

(4) ferromagnetic (χ>>1).

Diamagnetic materials effectively exclude an applied magnetic field, whereas paramagnetic materials effectively focus the applied magnetic field. Diamagnetic materials have an induced dipole moment in the presence of an external magnetic field. Atomic currents are created by orbiting electrons and the orbital motion in diamagnetic materials will change slightly such that the atomic current produces a weak magnetic field that opposes the external field. Paramagnetic materials contain atoms with an unpaired electron, which has an intrinsic magnetic moment that will tend to align with the external magnetic field. Therefore the applied magnetic field is slightly weaker around diamagnetic materials and slightly more intense near paramagnetic materials. Local magnetic field variations alter MR images and spectra by causing phase and frequency changes that can lead to distortions and signal loss.

Devices of the invention, including electrodes, infusion cannula, and interventional MRI instrumentation, are constructed with multiple layers or mixtures of metals or alloys that allow the diamagnetic behavior of some metals to combine with the paramagnetic behavior of others to produce the described result.

In accordance with the invention, experimental measurements, as well as modeling using Maxwell's equations, as further explained in the incorporated references, are carried out to obtain the optimal choice of geometry and metals, such that fluctuations in the local magnetic field near the interface between the device and surrounding material are minimized, thus reducing image and spectral distortions. In part, the invention achieves this result by providing devices whose magnetic susceptibility matches or nearly matches that of the material in which the device is inserted, wherein the devices may therefore be considered to be susceptibility-matched.

As an example, the invention enables the construction of susceptibility-matched recording electrodes for stimulation, and or acquiring electroencephalography (EEG) data before, during and after MR image and spectroscopy measurements, with a significant reduction in distortion of the resulting images and spectra. In accordance with the invention, these electrodes may further be incorporated into micro-electrode arrays.

Moreover, a susceptibility-matched metal cannula, in accordance with the invention, may be left in the target tissue before, during, and after MR imaging and spectroscopy, without creating significant distortion. More particularly, cannulas in accordance with the invention can be used to significantly reduce distortion of resulting images and spectra. In accordance with the invention, these cannulas may be used in vivo for infusions of therapeutic agents into tissue during MR sessions. Alternatively, the cannulas may be used for selective illumination of optically responsive cells in light activated cell channel control applications. The lumen of the cannula can be left hollow for non-optical uses. Alternatively, optical fibers may be inserted within the lumen, to thereby expose selected tissue regions to light. Because the cannula is constructed with metal, it may also function as a stimulating/recording electrode. Electrophysiology information, gathered from the recording function of the cannula of the invention, could be used to monitor physiology or target regions of tissue based on differences in electrophysiology.

The reduction in distortion provided by the invention enables electrodes to be positioned with greater precision, and for tissue near the electrodes to be properly characterized using MR imaging.

The invention may advantageously be used in at least the areas of brain monitoring, neural interfacing, electrical and optical stimulation, and control in Parkinson disease with deep brain stimulation, epilepsy, sleep disorders, multiple sclerosis, pain disorders, paralysis, cerebral palsy, depression, autism, and autonomic nervous system disorders.

In one embodiment of the invention, an electrode or cannula for use during magnetic resonance imaging (MRI) comprises a mixture of at least two diamagnetic and paramagnetic materials combined to form an electrode with at least a portion having the magnetic susceptibility of the tissue into which the electrode is to be inserted.

In another embodiment of the invention, an electrode or cannula for use during magnetic resonance imaging (MRI) comprises an elongated inner layer of paramagnetic material defining a radial thickness; an elongated outer layer of diamagnetic material, disposed in circumferential disposition to said inner layer, said outer layer defining a radial thickness relative to the radial thickness of said inner layer in a proportion which corresponds to an electrode having a magnetic susceptibility at the electrode surface that matches the magnetic susceptibility of a material in which the electrode is inserted.

In a further embodiment, an electrode or cannula for use during magnetic resonance imaging (MRI) comprises an elongated inner layer of platinum defining a radial thickness; an elongated outer layer of gold, disposed in circumferential disposition to said inner layer, said outer layer defining a radial thickness relative to the radial thickness of said inner layer of between about 6:19 and about 4:11; wherein the magnetic susceptibility of the electrode is near that of water.

In yet another embodiment, a method of reducing magnetic perturbations in magnetic resonance imaging, comprises providing at least one electrode or cannula having an elongated inner layer of platinum defining a radial thickness; an elongated outer layer of gold, disposed in circumferential disposition to said inner layer, said outer layer defining a radial thickness relative to the radial thickness of said inner layer of between about 6:19 and about 4:11; wherein the magnetic susceptibility of the electrode is near that of water; and wherein the perturbation to the magnetic field of the MRI device, as evident in the MR image, is substantially confined to the width of the electrode, when the electrode is positioned in material having a magnetic susceptibility near that of water during MR imaging.

In another embodiment of the invention, a method of reducing magnetic perturbations in magnetic resonance imaging, comprises providing at least one electrode or cannula having an elongated inner layer of platinum defining a radial thickness; an elongated outer layer of gold, disposed in circumferential disposition to said inner layer, said outer layer defining a radial thickness relative to the radial thickness of said inner layer of between about 6:19 and about 4:11; inserting at least one of the electrodes into material to be subjected to magnetic resonance imaging; and gathering electrical data from the at least one electrode while magnetic resonance imaging is taking place.

In an additional embodiment, an electrode or cannula to be placed into body tissue for use during magnetic resonance imaging (MRI) comprises an alloy of about 54 wt % Ag and about 46 wt % Pd, the exact wt % of Ag and Pd selected to approximate the magnetic susceptibility of the tissue into which the electrode is to be placed.

In variations of embodiments of the invention, the diamagnetic and paramagnetic materials are selected from the group consisting of: gold, platinum, silver, and palladium; the paramagnetic material is platinum, the diamagnetic material is gold, and the material has a magnetic susceptibility substantially that of water; the proportion of radial thickness of the inner layer to the radial thickness of the outer layer is between about 6:19 and about 4:11; the electrode, during use, has an end disposed within a material to be imaged, and wherein the end has a shaped end, wherein the shaped end produces a recognizable perturbation within a magnetic resonance image; the electrode or cannula has a polymeric coating; the electrode or cannula inner layer forms a lumen, and wherein the device may function as a cannula; the perturbation to the magnetic field of the MRI device, as evident in the MR image, is substantially confined to about the width of the electrode, when the electrode is positioned in material having a magnetic susceptibility near that of water during MR imaging.

In other variations of embodiment in accordance with the invention, a plurality of electrodes is provided in an array, positionable within body tissue; the electrode or cannula inner layer has a radial thickness of 6 μm, and an outer layer has a radial thickness of 19 μm; the electrode or cannula has an inner layer of platinum provided with a hollow lumen, thereby forming a cannula having a distal end having an aperture; and providing a fiber optic cable sized to be passed through a cannula of the invention, operative to transmit light through the aperture.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 (PRIOR ART) is a schematic illustration of the composition of a typical electrode of the prior art;

FIG. 2 (PRIOR ART) depicts an image of the brain, with the portion encircled in a white ellipse highlighting distortional artifacts near the ends of the electrodes, resulting from the use of prior art electrodes;

FIG. 3 (PRIOR ART) depicts the image of FIG. 2, wherein the white ellipse highlights the perceptual increase in dimensional width of the electrodes, as viewed longitudinally;

FIG. 4 (PRIOR ART) illustrates a simulation of a 50 μm tungsten electrode of the prior art, and associated perturbation to an external magnetic field, with a dashed circle indicating the actual circumference of the electrode end;

FIG. 5 illustrates a simulation of a 50 μm electrode in accordance with the invention, and associated diminished perturbation to an external magnetic field, with a dashed circle indicating the actual circumference of the electrode end;

FIG. 6 is a plot of integrate field perturbation for a simulated electrode in accordance with the invention, as the diameter of the binary core material (platinum) increases;

FIG. 7 depicts the plot of FIG. 6, with the vertical scale expanded;

FIG. 8 is a plot of calculated magnetic field change (AB) caused by a 50 μm wire in brain tissue, in a 200 μm field of view, comparing prior art electrodes, and electrodes in accordance with the invention;

FIG. 9. is an illustration of an electrode in accordance with the invention;

FIG. 10 is an illustration of a cannula in accordance with the invention;

FIG. 11 (PRIOR ART) illustrates a simulation of a 170 μm silica cannula of the prior art, and associated perturbation to an external magnetic field, with a dashed circle indicating the actual circumferential cross-section of the cannula;

FIG. 12 illustrates a simulation of a 170 μm cannula in accordance with the invention, and associated diminished perturbation to an external magnetic field, with a dashed circle indicating the circumference of the prior art cannula of FIG. 11;

FIG. 13 is a plot of integrate field perturbation for a simulated cannula in accordance with the invention, as the diameter of the binary inner material (platinum) increases;

FIG. 14 depicts the plot of FIG. 13, with the vertical and horizontal scales expanded;

FIG. 15 illustrates a simulation of a 50 μm binary wire electrode in accordance with the invention, the electrode provided with perpendicular cut at an end portion, and associated diminished perturbation to an external magnetic field;

FIG. 16 illustrates a simulation of a 50 μm binary wire electrode in accordance with the invention, the electrode provided with a 45 degree angle cut at an end portion, and associated diminished perturbation to an external magnetic field;

FIG. 17 is a plot of the field perturbation for the simulated electrode along the dashed line of FIG. 15;

FIG. 18 is a plot of the field perturbation for the simulated electrode along the dashed line of FIG. 16;

FIG. 19 illustrates a simulation of a 170 μm cannula in accordance with the invention, the cannula provided with perpendicular cut at an end portion, and associated diminished perturbation to an external magnetic field;

FIG. 20 illustrates a simulation of a 170 μm cannula in accordance with the invention, the cannula provided with a 30 degree angle cut at an end portion, and associated diminished perturbation to an external magnetic field;

FIG. 21 is a plot of the field perturbation for the simulated cannula along the dashed line of FIG. 19;

FIG. 22 is a plot of the field perturbation for the simulated cannula along the dashed line of FIG. 20;

FIG. 23A illustrates an alloy test ingot in accordance with the invention;

FIG. 23B is an MRI image of a test ingot comprising an alloy of 50:50 Zn—Al in accordance with the invention;

FIG. 23C is an MRI image of a test ingot comprising Aluminum;

FIG. 23D is an MRI image of a test ingot comprising Zinc;

FIG. 23E is an MRI image of a test ingot comprising an alloy of 50:50 Zn—Al in accordance with the invention, imaged at a higher-frequency bandwidth (lower frequency resolution) than the image of FIG. 23B;

FIG. 23F is an MRI image of a test ingot comprising Aluminum, imaged at a higher-frequency bandwidth (lower frequency resolution) than the image of FIG. 23C; and

FIG. 24 is a plot of volume magnetic susceptibility for varying weight percentages of Ag, for an alloy of Ag and Pd, and an overlay of electrical resistivities calculated in accordance with reference number 4, Marques et al., and reference number 5, Kemp et al., respectively.

### DETAILED DESCRIPTION OF THE INVENTION

In the description which follows, the particular embodiments described herein are not to be considered as limiting of the present invention.

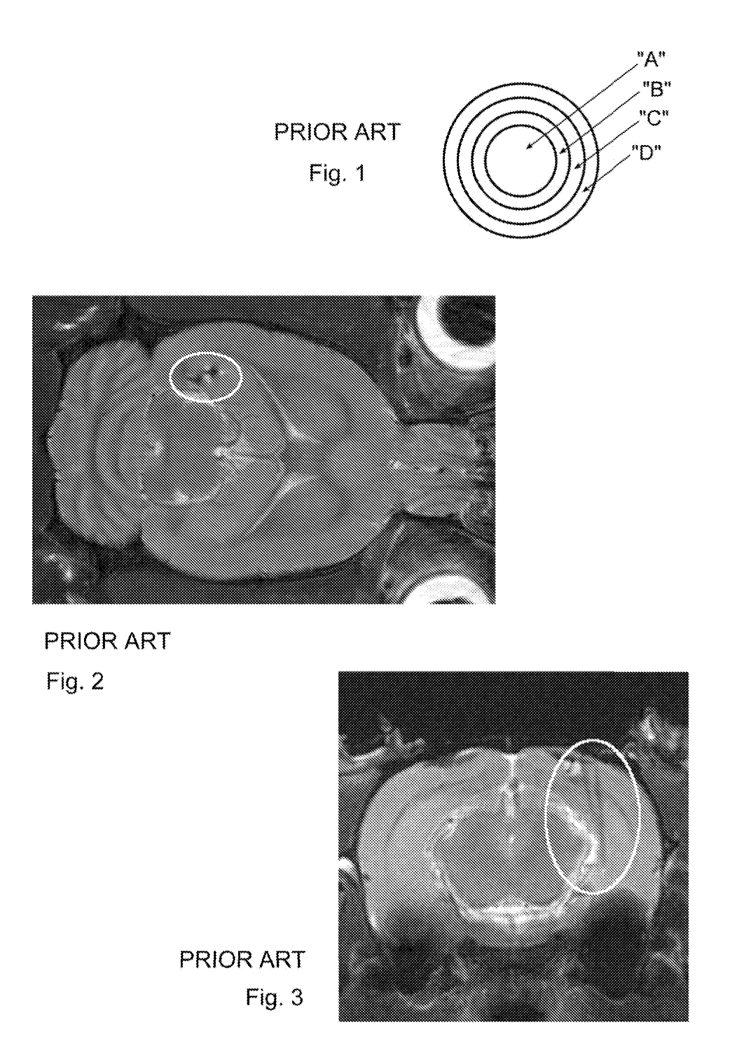

Prior art devices used during Magnetic Resonance Imaging (MRI) and MR spectroscopy (hereafter MR), that have magnetic susceptibility that is poorly matched to surrounding tissue, can produce perturbations in the local magnetic field, creating MR image and spectral distortions introduced near the interface between the device and the surrounding material. A schematic illustration of a typical prior art electrode is provided in FIG. 1, in which “A” represents a 50 μm diameter tungsten core; “B”, a 0.13-0.38 μm thick nickel flash; “C”, a 1.3 μm gold layer; and “D”, a 58-78 μm polyimide coating.

With reference to FIGS. 2-3, the chronic limbic epilepsy rat model requires the placement of chronic electrodes in a specific brain region. To minimize the impact, extremely small electrodes (e.g. 50 μm diameter wire) are used. However, electrodes within the brain of animals that are part of a longitudinal MR imaging study must be both “MRI compatible” (not highly permeable and do not move in the static magnetic field) and advantageously have appropriate susceptibility properties [1]. While in the MR magnet, these electrodes are not attached to an external measuring device (do not present a closed conductive loop), so no current is induced by the switched electric and magnetic fields, and the device is thus safe to remain in place during MRI. Electrodes have been introduced which are “MRI compatible” [2], but these electrodes distort the MR image to a considerable distance away from the electrodes [2]. In FIGS. 2-3, prior art “MRI-compatible” 50 μm diameter tungsten electrodes are used, which appear in T2-weighted 11.1 T MR images taken in vivo. As can be seen, the susceptibility-dependent shifts in the MR frequency distort the image as far as 0.35 to 1.1 mm away from the electrodes, visible within white ovals. Distortion near the electrode is visible as light and dark areas in FIG. 2. Distortion along the length of the electrodes is visible in FIG. 3, in which the electrodes appear much wider than their actual dimension.

In accordance with the invention, this effect is minimized by the selection of materials magnetic susceptibility-matched to brain tissue. Software tools were developed to simulate the magnetic properties, and to thereby facilitate the design of brain-tissue-susceptibility-matched electrodes in accordance with the invention. With electrodes in accordance with the invention, electrode position can be accurately determined and tissue near the electrodes can be more accurately characterized with MR imaging.

Devices of the invention, including electrodes, infusion cannula, and interventional MRI instrumentation, are constructed with multiple layers or mixtures of metals or alloys that allow the diamagnetic behavior of some metals to combine with the paramagnetic behavior of others to reduce variances in the local magnetic field, the latter of which can result in signal loss and affect the spatial encoding of the MR image resulting in image distortions.

Modeling is accomplished, in accordance with the invention, to obtain the optimal choice of geometry and metals, such that fluctuations in the local magnetic field near the interface between the device and surrounding material are minimized, thus reducing image and spectral distortions. In part, the invention achieves this result by providing devices whose magnetic susceptibility matches or nearly matches that of the target material, wherein the devices may therefore be considered to be susceptibility-matched.

To facilitate designing an electrode that eliminates the image artifact produced by the electrode shown and described with respect to FIGS. 2-3, the inventors developed a susceptibility analysis software system (SASS), written in IDL (ITT Visual Information Solutions, Boulder, Colo.), which calculates the expected perturbation to the static magnetic field surrounding a stimulation/recording electrode. The software uses the approach described in references [3] and [4], by Martinez-Santiesteban, et al., and Marques, J. P. and R. Bowtell, which references are incorporated herein by reference. The accuracy of the model was tested against empirical data, and demonstrated to be reliable.

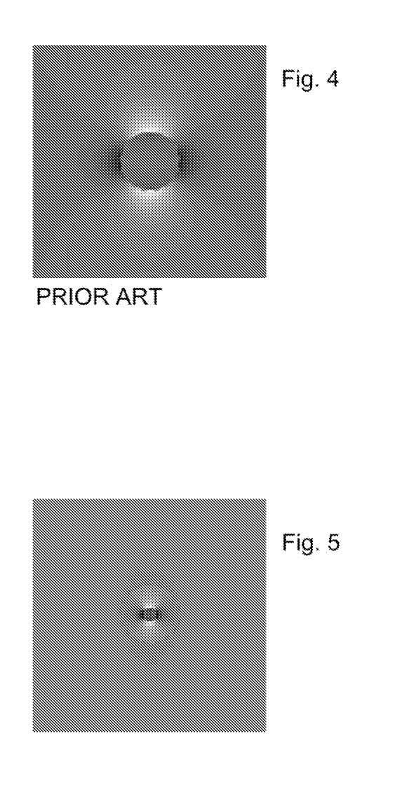

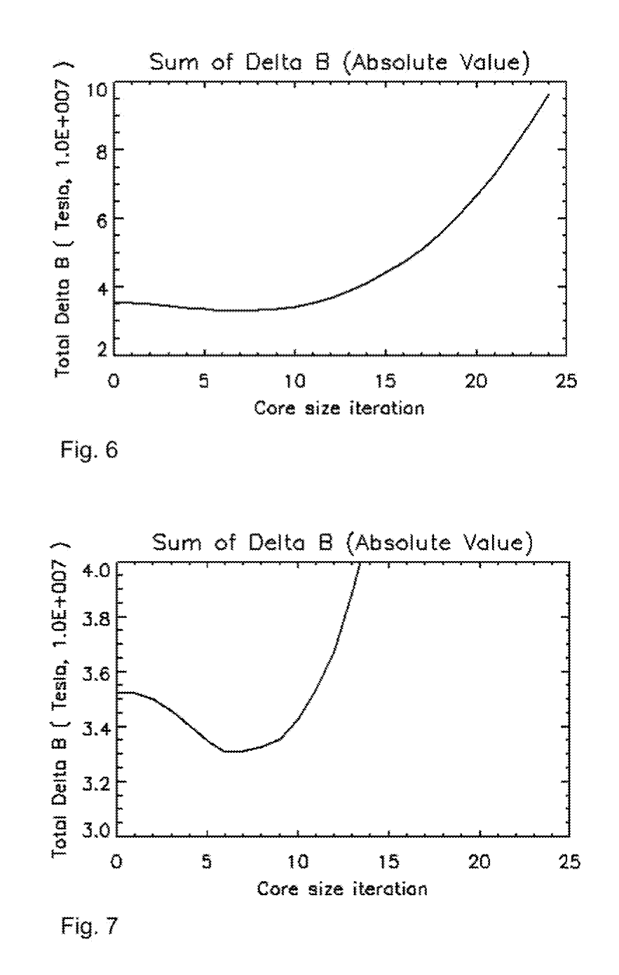

Using the software, the inventors simulated the perturbation to magnetic field surrounding an electrode of various materials, as may be seen with reference to FIGS. 4-7. FIG. 4 illustrates the simulation results for a prior art tungsten electrode, and FIG. 5 illustrates a binary-composite electrode of platinum and gold of the invention, the latter designed to match the susceptibility of brain tissue (−9.05 ppm). In these simulations, a static magnetic field of 11.1 Tesla was assumed, oriented perpendicular to an infinitely long cylindrical electrode (although a simulation of the field perturbation around finite objects, such as the electrode tips shown for example in FIGS. 2 and 3, is possible). The field of view was 200 μm and the electrode was 50 μm in diameter (shown as a dashed line in each figure). With reference to FIG. 6, to optimize the design, the diameter of the binary wire core radius was iterated, and the field perturbation was integrated over the area of the external field perturbation. The minimum external field perturbation can be seen in the expanded plot of FIG. 7. Since the field perturbation, external to the binary wire, is approximately minimized for a binary-composition wire with 6 μm radius platinum-core and 19 μm thick outer annulus of gold, this binary composite wire was chosen for the simulation of FIG. 5, in order to make a comparison to the prior art tungsten wire of FIG. 4.

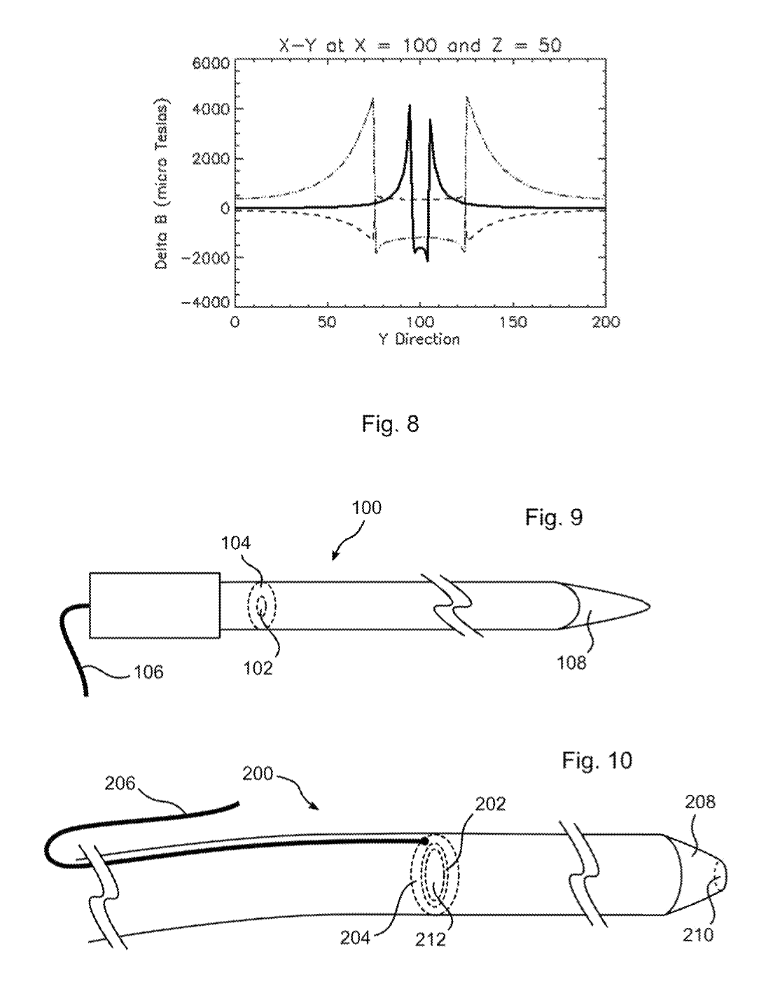

As can be seen in FIG. 5, the distortion is confined to an area smaller than the diameter of the electrode. With reference to FIG. 8, a plot is calculated of the magnetic field change (AB) caused by a 50 μm wire in brain tissue, in a 200 μm field of view, comparing prior art electrodes, and electrodes in accordance with the invention. More particularly, the dashed line illustrates a prior art tungsten wire, the dash-dot line a prior art gold wire, and the solid line a composite wire in accordance with the invention, having a gold annulus with a 19 μm outer radius over a platinum core with a 6 μm radius. As can be seen in the figure, the ΔB, or distortion of the electrode of the invention is confined within the dimensions of the electrode (50 μm), as compared with the prior art electrodes, which demonstrate distortion beyond the range of the plot, or greater than 100 μm in each direction.

Accordingly, the invention enables the construction of susceptibility-matched recording electrodes for acquiring electroencephalography (EEG) data before, during and after MR image and spectroscopy measurements, with a significant reduction in distortion of the resulting images and spectra. In accordance with the invention, these electrodes may further be incorporated into micro-electrode arrays.

An electrode 100 in accordance with the invention is illustrated in FIG. 9, having a core 102 formed with platinum, and an outer layer 104 formed with gold. A lead 106 extends from electrode 100, and may be connected to core 102, outer layer 104, or both. Tip 108 may be coated with gold layer 104, or core 102 may be exposed. All or a portion of the electrode may be coated with polyimide, Nylon, or other coating materials known and used in the art. The coating serves at least two functions in the context of an electrode to be placed in body tissue: (1) the wire must be electrically insulated, with only the tip exposed, and (2) the coating minimizes the biological reaction of the tissue to the presence of the metal. Electrode 100 is illustrative of a typical electrode, however it should be apparent to one skilled in the art that the invention may be adapted to other electrode designs. For example, a bipolar electrode may be formed of two electrodes 100, in a manner known in the art.

Further in accordance with the invention, a susceptibility-matched metal infusion cannula may be left in the target tissue during MR imaging and spectroscopy, without creating significant distortion. In accordance with the invention, it is advantageous for the region or portion of the electrode in proximity to the area being imaged to be susceptibility matched. Portions sufficiently far from the area being imaged do not need to be closely matched, provided no significant perturbation is imposed upon the area being imaged. In accordance with the invention, these cannulas may be used in vivo for infusions of therapeutic agents into tissue during MR sessions. Alternatively, the cannulas may be used for selective illumination of optically responsive cells in light activated cell channel control applications. The lumen of the cannula can be left hollow for non-optical uses.

Alternatively, optical fibers may be inserted within the lumen, to thereby expose selected tissue regions to light. Because the cannula is constructed with metal, it may also advantageously function as a stimulating/recording electrode. Electrophysiology information, gathered from the recording function of the cannula of the invention, could be used to monitor physiology or target regions of tissue based on differences in electrophysiology.

In accordance with the invention, an advantageous proportion of core platinum to outer layer gold has been found to be 6:19. Thus, a 25 μm electrode has a platinum core having a radial thickness of 3 μm, and an outer gold layer having a radial thickness of 9.5 μm. Correspondingly, a 50 μm electrode has a platinum core having a radial thickness of 6 μm, and an outer gold layer having a radial thickness of 19 μm. Of course, these values may vary and still be effective, as may be seen in the illustration of FIGS. 6-7. Additional, the purity of materials, and the particular tissue susceptibility also may result in a different proportion than the 6:19 proportion of the invention.

In accordance with an alternative embodiment of the invention, an electrode formed with a mixture or alloy of silver (Ag, which is diamagnetic) and palladium (Pd, which is paramagnetic) having a composition with about 54 wt % Ag and about 46 wt % Pd, whereby the magnetic susceptibility of the electrode is near that of water. In accordance with the invention, the wt % of alloy may vary by 10%, wherein the exact ratio is selected to best match the magnetic susceptibility of the tissue into which the electrode is to be placed.

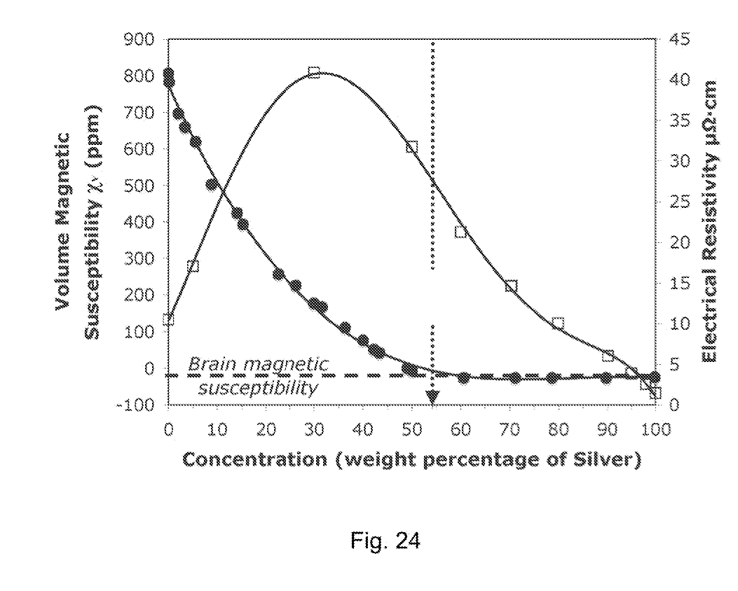

The foregoing is illustrated in FIG. 24, which is a plot of volume magnetic susceptibility and electrical resistivities for varying weight percentages of Ag, for an alloy of Ag and Pd, calculated in accordance with Marques, et al. (Ref 4, below), and Kemp, et al. (Ref 5, below), respectively.

Further in accordance with the invention, alloys of the invention may be cast and heat treated in order to obtain a microstructure in the alloy that will produce a magnetic susceptibility that will match that of the tissue into which a device of the invention is inserted. The alloy obtained may then be formed into a single or multilayer layer wire or tube by drawing, swaging, or machining, to create a geometry or final configuration as described herein for devices of the invention, such as an electrode or cannula or the like, operative to yield a reduced distortion or perturbation in an image or spectra.

Further in accordance with the invention, alloys of the invention may be formed into a single or multi-layer electrode or cannula. For example, a biocompatible single layer electrode may be formed from an alloy of about 54 wt % Ag and about 46 wt % Pd, which is calculated to produce, in accordance with the invention, a diminished perturbation to an external magnetic field as compared to electrodes of the prior art. In accordance with the invention, a variance of about 10 wt % for either alloy is calculated to produce acceptable results, with a significant loss of quality, or increase in perturbation, expected beyond this variance.

FIG. 10 illustrates a cannula 200 in accordance with the invention, having a lumen 212 in fluid communication with an aperture 210, formed in tip 208. An inner layer 202 defining lumen 212 is formed of platinum, and an outer layer 204 is formed of gold. Means not shown, such as a tube, are connected to lumen 212 in order to pass material or instruments, including fiber optic strands, into lumen 212. Alternatively, lumen 212 may extend to pass outside the body, whereby materials may be admitted directly. A lead 206 extends from cannula 200, and may be connected to inner layer 202, outer layer 204, or both. Tip 208 may be coated with gold layer 204, or inner layer 202 may be exposed. As with electrodes, cannula 200 may be partially or completely coated with polyimide, Nylon, or similar materials as known in the art. Cannula 200 is illustrative of a typical cannula, however it should be apparent to one skilled in the art that the invention may be adapted to other cannula designs.

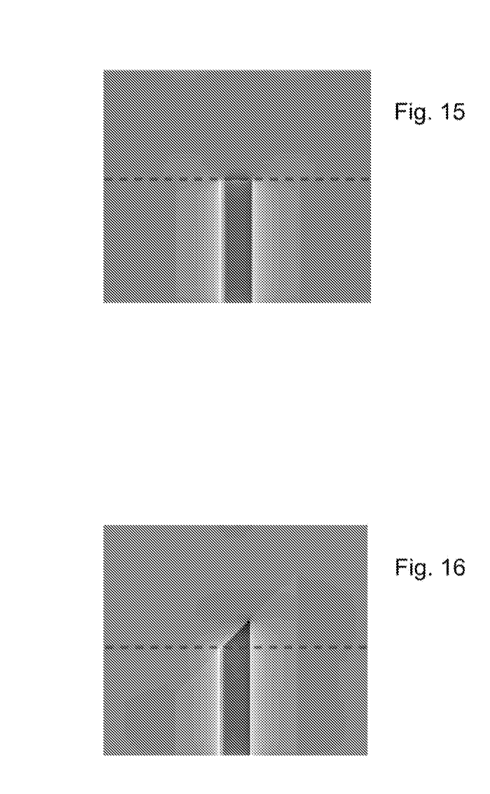

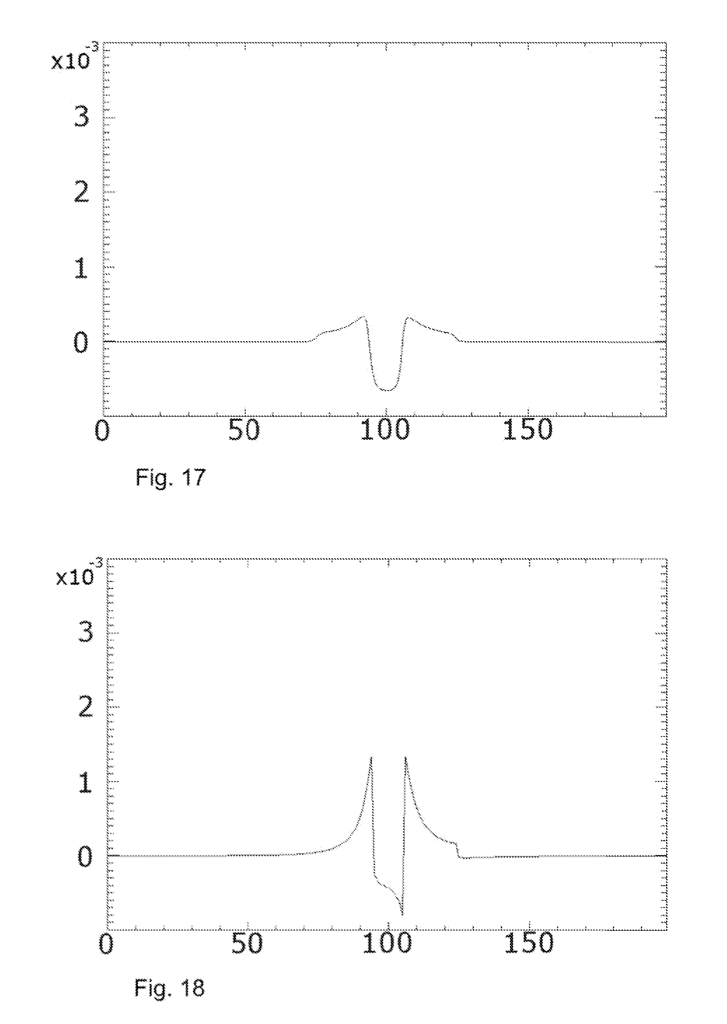

The reduction in distortion provided by the invention enables electrodes to be positioned with greater precision, and for tissue near the electrodes to be properly characterized using MR imaging. For example, electrodes that are susceptibility-matched to brain tissue in accordance with the invention, may be chronically implanted into the ventral hippocampus for stimulation and recording. The prior art 50 μm diameter tungsten electrodes distort the MR image to an extent whereby it is not possible to accurately locate the electrode or visualize tissue within a few mm of the electrode in MR images. Composite wire of the invention as described herein may be fabricated, at the time of this writing, by Fort Wayne Metals, of Fort Wayne, Indiana, using their “drawn filled tube” process. The platinum-gold composite wire electrode is calculated to be invisible or substantially invisible in the MR image; however, the tip is expected to be visible due to the end effects near the cut end of the wire composite. It is possible to design the tip of the wire to be specifically visible, without overly perturbing the tissue image near the tip. Specifically by shaping the tip to have desirable features (as shown in FIGS. 15 and 16 with magnetic susceptibility effects as illustrated in FIGS. 17 and 18).

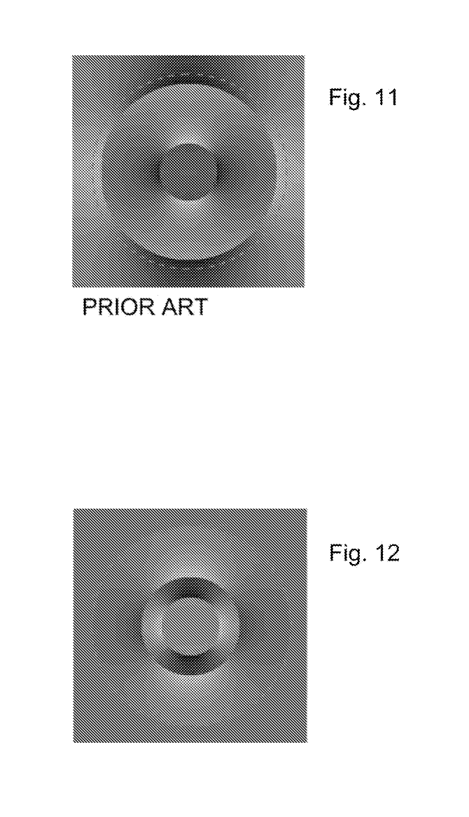

Referring now to FIGS. 11-14, FIG. 11 depicts results of a simulation of cannula susceptibility perturbation to an external magnetic field, for a prior art cannula. The prior art cannula consists of a 50 μm diameter lumen surrounded by a fused-silica layer having a radial thickness of 50 μm, where the fused-silica layer is then covered by a polyimide coating having a radial thickness of 10 μm. This 170 μm diameter cross-section is shown as a dashed line in FIGS. 11-12.

An optimized binary platinum-gold cannula in accordance with the invention is shown in FIG. 12, having a 170 μm diameter cross-section, including 50 μm diameter lumen, an inner layer of platinum having a 16 μm radial thickness, and an outer layer of gold having a 44 μm radial thickness.

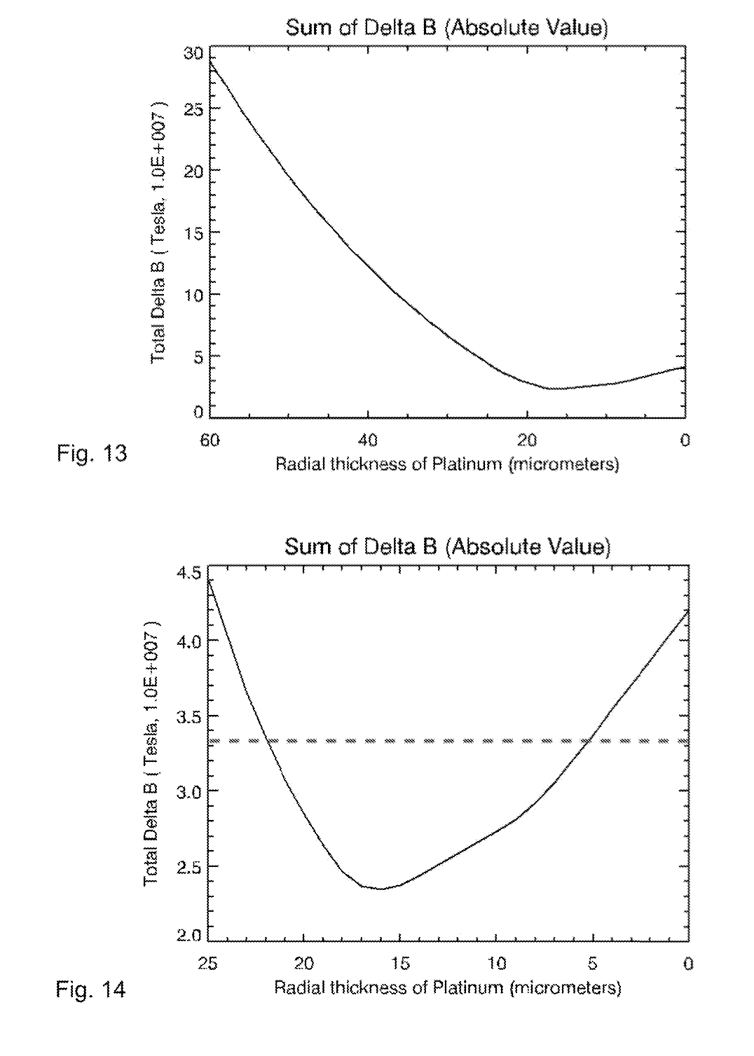

FIG. 13 illustrates a plot of the integrate field perturbation, as the radius of the platinum binary inner material is increased. FIG. 14 illustrates the plot of FIG. 13, with expanded vertical and horizontal scales. A horizontal dashed line indicates the integrated field perturbation of a fused-silica cannula of the same size. As may be seen, the cannula in accordance with the invention has a lower integrate field perturbation and the desired conductive properties of a metal cannula. For the cannula of the invention, an advantageous ratio of paramagnetic material, here platinum, to diamagnetic material, here gold, is determined to be 4:11.

Referring now to FIGS. 15-18, FIG. 15 depicts results of a simulation of a 50 μm electrode in accordance with the invention, the electrode having a platinum core having a radial thickness of 6 μm, and an outer layer of gold having a radial thickness of 19 μm. The electrode has a perpendicular cut to form the tip at the distal end. FIG. 16 depicts the electrode of FIG. 15, now provided with an angular cut at a distal end. In this embodiment, a cut of 45 degrees has been formed. Results indicating the perturbation of an external magnetic field are shown in FIGS. 17-18, for the embodiments of FIGS. 15 and 16, respectively, and where the vertical axis is units of Tesla. The perturbation is calculated, for each electrode embodiment, along the location indicated by the dashed lines in FIGS. 15 and 16. Although the perturbation appears more significant in the cut end embodiment of FIG. 16, it can be seen that shaped electrodes may still be fashioned to exhibit reduced perturbation as compared to electrodes of the prior art but with desirable tip susceptibility effects so that the tip is emphasized in the MR image.

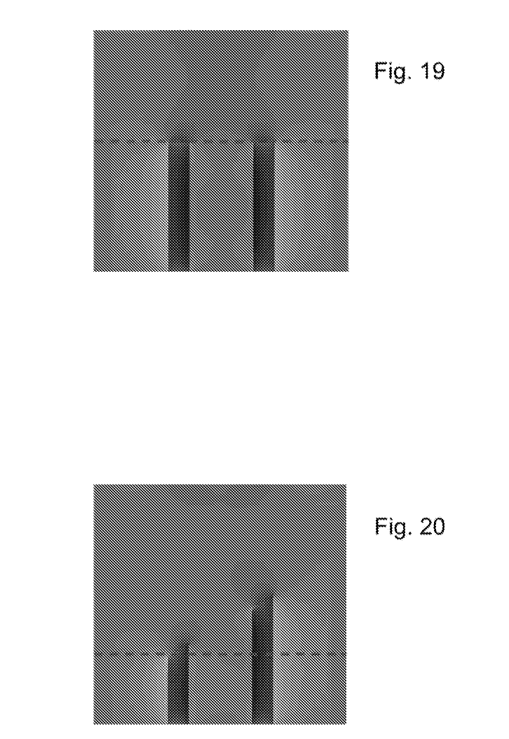

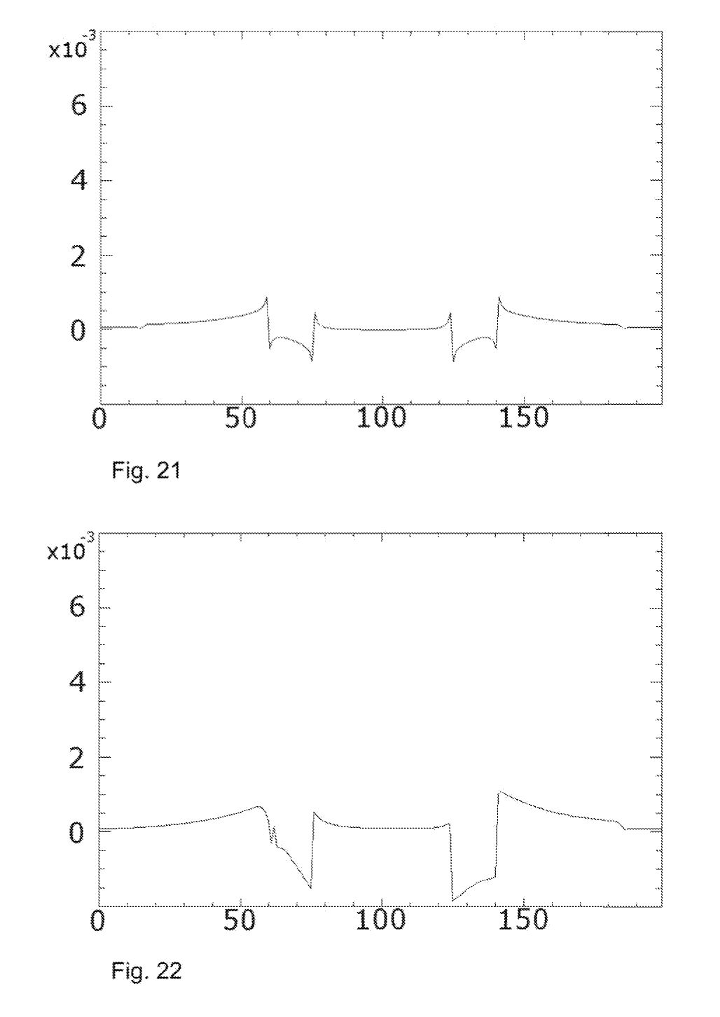

With reference to FIGS. 19-22, FIG. 19 depicts results of a simulation of a 170 μm cannula in accordance with the invention, the cannula having a lumen with a radial thickness of 25 μm, an inner layer of platinum having a radial thickness of 16 μm, and an outer layer of gold having a radial thickness of 44 μm, defining a ratio of radial thickness of 4:11, with a perpendicular cut at the distal end, at the position indicated by the dashed line. FIG. 20 depicts the cannula of FIG. 19, now provided with an angular cut at a distal end. In this embodiment, a cut of 30 degrees has been formed. Results indicating the perturbation of an external magnetic field are shown in FIGS. 21-22, for the embodiments of FIGS. 19 and 20, respectively, and where the vertical axis is units of Tesla. The perturbation is calculated, for each electrode embodiment, along the location indicated by the dashed lines in FIGS. 19 and 20.

The magnetic susceptibility perturbation, at the cut end of the cannula, can be designed to exhibit a characteristic shape, for example as shown in the figures.

This enables the end of the device to have a predictable and readily recognized appearance in the magnetic resonance image, which corresponds to the cut end of the device.

Additionally, an angle cut in particular may advantageously be formed in order to create a sharp edge on the leading end surface of the electrode or cannula, allowing for easier penetration into the material to be imaged.

It should be understood that cannulas and electrodes in accordance with the invention may be fabricated with different dimensions than described herein, as needed for particular requirements. While proportions of diamagnetic to paramagnetic material in these examples are about 4:11 for the described cannula of the invention, and about 6:19 for the described electrode of the invention, factors affecting the optimum ratios for a particular embodiment include the actual dimensions of the device, the purity and types of materials, and the nature of material into which the devices are inserted. Accordingly, some deviation from these ratios is to be expected, for example up to about 10% variation.

Moreover, by varying the diamagnetic and paramagnetic materials used to design a device of the invention, the overall size can be controlled as long as the material properties allow the fabrication of a device of the desired size and shape for the intended application. The circular cross-section in this embodiment of the invention is not the only possible shape, and other shapes, with appropriate proportions of diamagnetic and paramagnetic materials as described herein, should be considered to be within the scope of the invention, to one skilled in the art. Thus, the invention specifically contemplates fabrication of devices using other diamagnetic and paramagnetic materials besides gold and platinum, and of other shapes and sizes of devices, using the methods of the invention described herein.

As with the electrode of the invention, it can be seen in FIG. 12 that the magnetic field perturbation is confined to a cross-sectional area smaller than the circumference of the cannula. As compared to the prior art, this enables more accurate determination of the cannula position, and better characterization of tissue near the cannula, when using MR imaging.

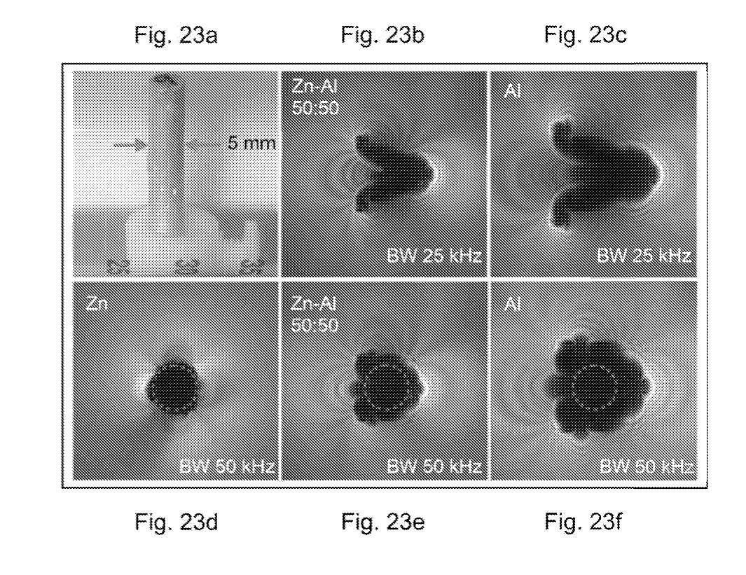

FIGS. 23A-23F illustrate an electrode of the invention, as well as single material electrodes, imaged with MRI. While the examples of FIGS. 23B and 23E illustrate the use of a Zinc (Zn)-Aluminum (Al) alloy of 50:50, or equal weight percentages, it should be understood that this alloy is not deemed particularly biocompatible for use in the brain. Rather, the materials were selected due to the relative ease of alloy production, to demonstrate an ability to effectively produce a device in accordance with the invention and to correctly measure a modification to magnetic susceptibility, and an ability to calculate the expected susceptibility.

With reference to FIG. 23A-F, three 5 mm diameter, 25 mm long ingots of zinc (Zn) and aluminum (Al) were prepared in the following proportions: pure Zn, 50:50 Zn—Al, and pure Al. Zn and Al were selected because they are readily available materials, they have opposite volume (magnetic) susceptibility, and can be combined as a solid solution alloy of the type described elsewhere herein. To produce the MR images, each ingot was positioned in a 30 mm diameter tube filled with tap water, and measured MR images of each ingot were performed in a device having a 11.1 T magnet, at 470 MHz. Each ingot was oriented perpendicular to the direction of the 11.1 T field. All images were measured with a gradient echo sequence using a recovery time of 300 ms, and an echo time of 4.5 ms, in a single 1 mm thick slice, having an in plane resolution of 0.2 mm, in a matrix of 150×150 data points.

The images in FIGS. 23B-F illustrate the sensitivity of MR imaging to distortions caused by the difference between the metal susceptibility and the susceptibility of the surrounding material (e.g., the water). FIGS. 23D-F illustrate the level of image distortion by indicating the actual size of the 5 mm diameter ingot by a dashed circle. In addition, the methods by which the MR image is measured affects the level of distortion visible in the image. For example, in FIGS. 23A-C, the frequency-encoded bandwidth (BW) was set to 25 kHz, and in FIGS. 23D-F, the images were measured with a 50 kHz BW. With an image matrix size of 150×150, the frequency resolution in the image was 167 Hz/pixel (0.355 ppm/pixel) for a BW of 25 kHz, and 333 Hz/pixel (0.709 ppm/pixel) for a BW of 50 kHz. As the volume susceptibility of Zn is approximately 12.5 ppm (diamagnetic) and the volume susceptibility of Al is 20.7 ppm (paramagnetic), relatively small changes in the measuremennt frequency bandwidth causes a significant change in the image distortion, due to the metal susceptibility being much larger than frequency resolution for either image acquisition. FIGS. 23A-F illustrate an ability to produce a diamagnetic/paramagnetic alloy in accordance with the invention, as well as an ability to perform susceptibility measurements. The figures further demonstrate the sensitivity of MR images to distortions caused by an imbalance in magnetic susceptibility.

It should be understood that while a combination of Ag and Pd, or Au and Pt have been illustrated, in accordance with the invention, a mixture of at least two of any of these or other diamagnetic and paramagnetic materials may be combined in accordance with the invention to produce an alloy and or layered combination with magnetic susceptibility matching body tissue into which devices of the invention are to be inserted. The particular combinations of materials suggested herein are selected for advantageous combination, particularly as alloys, from a metallurgical perspective, and for their biocompatibility. For example, an alloy of Au and Pd could be calculated to produce a desired magnetic susceptibility; however, from a metallurgical perspective, the combination is not as straightforward to produce as the alloy proposed.

It should further be understood that more than two paramagnetic and diamagnetic materials may be combined in layers or alloys, in accordance with the invention, to produce a device of the invention having a target magnetic susceptibility.

The present invention contemplates that electrodes or cannulas of the invention may be used either connected or not connected from electrical or electronic equipment, or may be provided by themselves, or incorporated into an implant or instrument.

MR compatible, susceptibility-matched neural interface sensors of the invention may be used to enable sensing and or stimulation of central and peripheral nervous systems extracellular electrical activities from individual neurons, groups of neurons, neural ensembles, and local field potentials. A MR compatible susceptibility-matched neurophysiologic sensing interface would be applicable for sensing and interacting with central nervous system brain and peripheral nerve neurophysiologic activities in both normal and pathological states such as epilepsy, Parkinson disease, particularly for deep brain stimulation, depression, stroke, psychiatric disorders, multiple sclerosis, paralysis, and cerebral palsy. A MR compatible susceptibility-matched neural interface will allow for precise targeting of an electrode(s) into distinct brain regions.

Alternatively, electrodes, cannulas, and other devices in accordance with the invention may be used in studying, diagnosing, and treating other areas of the body besides the nervous system, for example the circulatory, digestive, endocrine, immune, lymphatic, muscular, reproductive, respiratory, skeletal, and urinary systems.

It should further be understood that those skilled in the art may fabricate instruments and devices in accordance with the invention, which may advantageously be used during interventional MRI, to enable medical procedures to be carried out while MR imaging is taking place. Instruments and devices fabricated in accordance with the invention would have, at least, the advantages of magnetic compatibility of the “first kind” and the “second kind”, as described above, in addition to other advantages described herein.

Further, electrodes, cannula and other devices in accordance with the invention may be used in studying, developing, and controlling industrial processes that require highly homogeneous, undisturbed magnetic fields. This may be appropriate, for example, in a chemical or materials processing application.

All references cited herein are expressly incorporated by reference in their entirety. In addition, unless mention was made above to the contrary, it should be noted that all of the accompanying drawings are not to scale. There are many different features to the present invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention.

The following References are incorporated herein by reference:

1. Schenck, J. F., The role of magnetic susceptibility in magnetic resonance imaging: MRI magnetic compatibility of the first and second kinds. Medical Physics, 1996. 23(6): p. 815-850.

2. Jupp, B., et al., MRI compatible electrodes for the induction of amygdala kindling in rats. J Neurosci Methods, 2006. 155(1): p. 72-6.

3. Martinez-Santiesteban, F. M., et al., Magnetic field perturbation of neural recording and stimulating microelectrodes. Physics in Medicine and Biology, 2007(8): p. 2073.

4. Marques, J. P. and R. Bowtell, Application of a Fourier-based method for rapid calculation of field inhomogeneity due to spatial variation of magnetic susceptibility. 2005. Concepts in Magnetic Resonance Part B, 25B(1): p. 65-78.

5. Kemp, W. R. G., Klemens, P. G., Sreedhar, A. K., and White, G. K., The Thermal and Electrical Conductivity of Silver-Palladium and Silver-Cadmium Alloys at Low Temperatures. Proceedings of the Royal Society of London, Series A. Mathematical and Physical Sciences. 1955, 233(1195): p. 480-493.

1. An electrode or cannula for use during magnetic resonance imaging (MRI), the electrode comprising a mixture of at least two diamagnetic and paramagnetic materials combined to form an electrode with at least a portion having the magnetic susceptibility of the tissue into which the electrode is to be inserted.

2. An electrode or cannula for use during magnetic resonance imaging (MRI), the electrode comprising:
an elongated inner layer of paramagnetic material defining a radial thickness;
an elongated outer layer of diamagnetic material, disposed in circumferential disposition to said inner layer, said outer layer defining a radial thickness relative to the radial thickness of said inner layer in a proportion which corresponds to an electrode having a magnetic susceptibility at the electrode surface that matches the magnetic susceptibility of a material in which the electrode is inserted.

3. An electrode or cannula for use during magnetic resonance imaging (MRI), the electrode comprising:
an elongated inner layer of platinum defining a radial thickness;
an elongated outer layer of gold, disposed in circumferential disposition to said inner layer, said outer layer defining a radial thickness relative to the radial thickness of said inner layer of between about 6:19 and about 4:11;
wherein the magnetic susceptibility of the electrode is near that of water.

4. A method of reducing magnetic perturbations in magnetic resonance imaging, comprising:
providing at least one electrode or cannula as set forth in claim 3;
the at least one electrode or cannula in a material,
wherein the perturbation to the magnetic field of the MRI device, as evident in the MR image, is substantially confined to the width of the electrode, when the electrode is positioned in material having a magnetic susceptibility near that of water during MR imaging.

5. A method of reducing magnetic perturbations in magnetic resonance imaging, comprising:
providing at least one electrode or cannula having
an elongated inner layer of platinum defining a radial thickness;
an elongated outer layer of gold, disposed in circumferential disposition to said inner layer, said outer layer defining a radial thickness relative to the radial thickness of said inner layer of between about 6:19 and about 4:11;
inserting at least one of the electrodes into material to be subjected to magnetic resonance imaging;
gathering electrical data from the at least one electrode while magnetic resonance imaging is taking place.

6. An electrode or cannula to be placed into body tissue for use during magnetic resonance imaging (MRI), the electrode comprising an alloy of about 54 wt % Ag and about 46 wt % Pd, the exact wt % of Ag and Pd selected to approximate the magnetic susceptibility of the tissue into which the electrode is to be placed.

7. The electrode or cannula of claim 1, wherein the diamagnetic and paramagnetic materials are selected from the group consisting of: gold, platinum, silver, and palladium.

8. The electrode or cannula of claim 1, wherein the paramagnetic material is platinum, the diamagnetic material is gold, and the material has a magnetic susceptibility substantially that of water.

9. The electrode or cannula of claim 1, wherein the proportion of radial thickness of said inner layer to the radial thickness of said outer layer is between about 6:19 and about 4:11.

10. The electrode or cannula of claim 1, wherein the electrode, during use, has an end disposed within a material to be imaged, and wherein said end has a shaped end, wherein said shaped end produces a recognizable perturbation within a magnetic resonance image.

11. The electrode or cannula of claim 1, further comprising a polymeric coating.

12. The electrode or cannula of claim 1, wherein the inner layer forms a lumen, and wherein the device may function as a cannula.

13. The electrode or cannula of claim 1, wherein the perturbation to the magnetic field of the MRI device, as evident in the MR image, is substantially confined to about the width of the electrode, when the electrode is positioned in material having a magnetic susceptibility near that of water during MR imaging.

14. The electrode or cannula of claim 1, wherein a plurality of electrodes is provided in an array, positionable within body tissue.

15. The method of claim 5, wherein the inner layer has a radial thickness of 6 μm, and an outer layer has a radial thickness of 19 μm.

16. The electrode or cannula of claim 3, wherein the inner layer of platinum is provided with a hollow lumen, thereby forming a cannula having a distal end having an aperture.

17. The cannula of claim 12, further including the step of providing a fiber optic cable sized to be passed through the cannula, operative to transmit light through the aperture.

18. The electrode or cannula of claim 2, wherein the electrode, during use, has an end disposed within a material to be imaged, and wherein said end has a shaped end, wherein said shaped end produces a recognizable perturbation within a magnetic resonance image.

19. The electrode or cannula of claim 2, wherein the inner layer forms a lumen, and wherein the device may function as a cannula.

20. The electrode or cannula of claim 2, wherein the perturbation to the magnetic field of the MRI device, as evident in the MR image, is substantially confined to about the width of the electrode, when the electrode is positioned in material having a magnetic susceptibility near that of water during MR imaging.
